(19) United States
(12) Plant Patent Application Publication
(10) Pub. No.: US 2009/0038037 P1
(43) Pub. Date: Feb. 5, 2009

(54) Floribunda rose plant named 'MORabundant'

(75) Inventor: Ralph S. Moore, Visalia, CA (US)

Correspondence Address: GARY S. COBB;COTTAGE HILL NURSERY, INC., 9960 PADGETT SWITCH ROAD, IRVINGTON, AL, 36544, US

(73) Assignee: Cottage Hill Nursery, Inc., Irvington, AL (US)

(21) Appl. No.: 11/888,874

(22) Filed: Aug. 3, 2007

(51) Int. Cl. A01H 5/00

(52) U.S. Cl. PLT/148

(57) ABSTRACT

A new and distinct cultivar of rose plant of the floribunda class, of hardy, sturdy, bushy, much branched habit substantially as illustrated and described, characterized by buds and flowers, basically pink, the bud being pointed in the early stage, opening from light pink center to medium pink on outer petals, with the open flower being double and rounded with 40 or more petals; and further characterized by a plant of compact, vigorous, upright shape with main stems and shoots of less than average thorniness, the said plant being easy to propagate from soft-wood cuttings with an abundance of medium, semi-glossy foliage, the flowers being usually borne in medium clusters on medium length stems. Ralph Moore originated this new and distinct rose at his nursery located in Visalia, Calif., for the approval and subsequent sale to Cottage Hill Nursery of Irvington, Ala., who is now filing this Plant Patent document.

### CLASSIFICATION

The present invention relates to a new Rosa Floribunda plant (rosa spp.).

### VARIETY DENOMINATION

The new plant has the varietal denomination ‘MORabundant’.

### BACKGROUND OF THE INVENTION

This new and distinct cultivar of floribunda rose was originated by Ralph Moore, located at Visalia, Calif., as a result of a cross between ‘MORdora” (‘Sequoia Ruby’, miniature rose climber) as the seed parent and ‘seedling’ (Golden Gardens×Strawberry Ice) as the pollen parent.

### SUMMARY OF THE INVENTION

The present invention relates to a new and distinct cultivar of hardy, bush type rose plant of the floribunda class. The cultivar is primarily characterized as to novelty by its pink color and double flowers. The cultivar is further characterized by: an abundance of bloom, with flowers usually borne in clusters (sometimes singly) to a stem; slight fragrance; a plant of vigorous, rounded to compact, bushy growth shape, with semi-glossy foliage with elliptical leaflets; the buds and flowers open from light pink from center to medium pink outside tip petals and medium pink on reverse side of petal blooms; a plant which grows and blooms satisfactorily both in greenhouse and outdoors; with repeat bloom, is good for growing in pots, and provides excellent garden decoration as well as cut flowers.

The new cultivar has been successfully asexually reproduced by budding as well as by cuttings; the reproductions have run true in all respects. The photograph illustrates the cultivar including the foliage, buds and flowers.

### BRIEF DESCRIPTION OF THE DRAWING

Plant part coloration illustrated is as true as reasonably possible to attain in photographic illustrations of this type. The accompanying photographs illustrate specimens of the new variety and show the flowering thereof from bud to full bloom.

FIG. 1. shows open flower with buds and foliage

FIG. 2. shows open flower and foliage

FIG. 3. shows canes and foliage

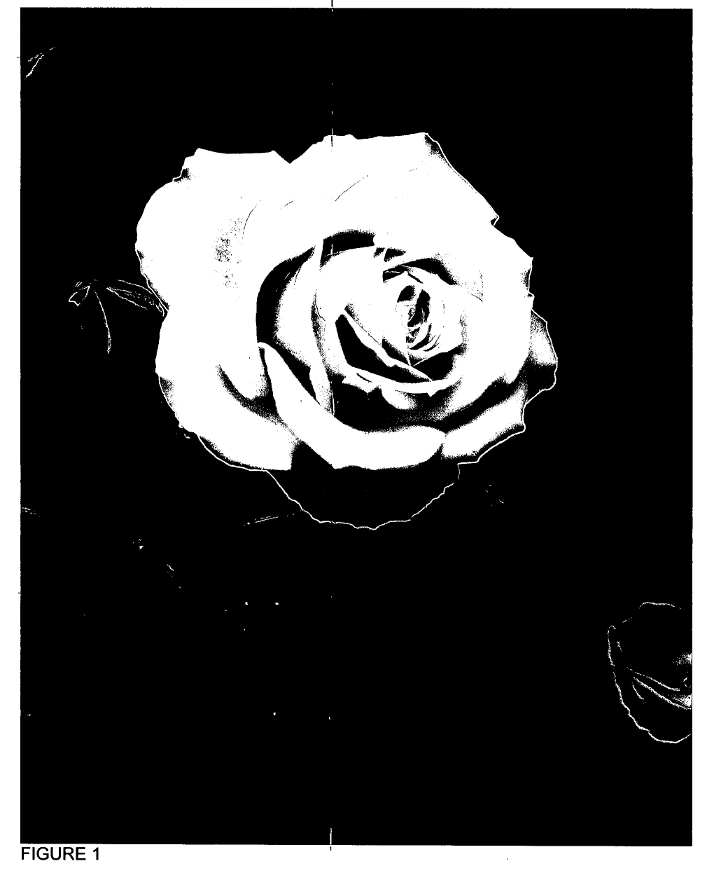

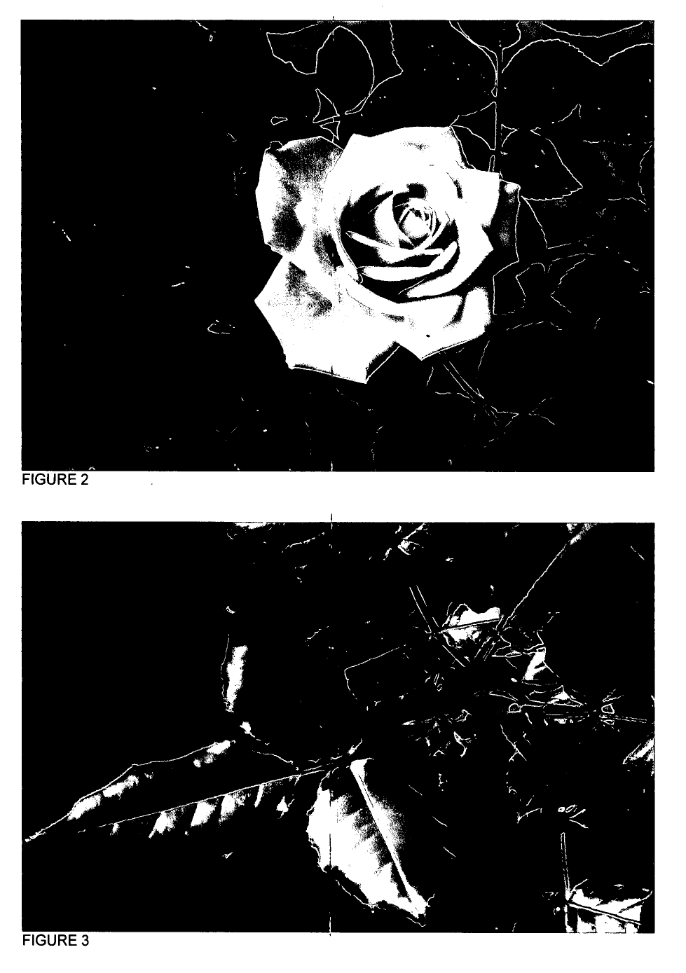

### DETAILED BOTANICAL DESCRIPTION OF THE PLANT

Referring now specifically to the new and distinct cultivar of floribunda rose plant, the following is a detailed description thereof in outline; all major color plate identifications being by reference to the R.H.S. Colour Chart (Royal Horticultural Society) except where common terms of color definition are employed.
Flower:
   Type.—Hardy; bushy, upright; seedling; greenhouse; outdoors; cut flowers; garden decoration; potted plant.
   Class.—Floribunda. Cultivar identification: ‘MORabundant’ (commercial synonym to be determined at a later date).
   Flowers borne.—Usually in loose clusters, sometimes singly, on sturdy, wiry stems.
   Quantity of bloom.—Abundant both outside and inside greenhouse.
Bud:
   Peduncle.—Medium length; sturdy; erect; medium green, near Green Group 138A, smooth.
   Before calyx breaks.—Size-medium.
   Form.—Pointed, with several small foliaceous appendages on surface of the bud; with foliaceous parts extending beyond the tip of the bud equal to one half or more than one half of its length.
   Color.—Medium green, near Green Group 138A, occasionally tinted with bronze (outdoors).
   As calyx breaks.—color — light pink, near Red-purple Group 62D to medium Pink.
   Opening.—Opens well in all weather.
Bloom:
   Size when fully open.—Large, averaging about 4 inches.
   Petalage.—Double — averaging about 40 petals, arranged regularly plus several small petaloids.
   Form.—Full, high centered at first, becoming rounded with petals rolled loosely outward and downward. Fully opened flowers usually show stamens (not shown in photo).
   Sepals.—single and reflexed.
   Behavior.—Opens well in all weather; spent petals or flowers drop off.
   Flower longevity.—plant in pot — 4 to 6 days or longer. Cut flowers at living room temperature — 4 to 5 days.
Petals: Fairly thick; satiny on inside and on outside surface.
   Shape.—Outside petals: broad orbicular, nearly round with small acute tip.
   Intermediate.—broad orbicular, rounded or nearly round with short acute tip.
   Inside.—broad orbicular, with rounded or acute tip.
   Color.—(Newly opening flower from plant grown in plastic greenhouse in December 2006, Visalia, Calif. (Photograph taken Dec. 2006)).
   Outside petal.—Outside surface — Pink near Red-Purple Group 62A blending into yellow at petal base.
   Inside surface.—near Red-Purple group 62D blending into yellow at petal base.
   Intermediate petal.—Outside surface similar to outside petal; inside surface similar to intermediate petal.
   Inner petal.—Outside surface similar to intermediate petal; inside surface similar to intermediate petal.
General color effect:
   Newly opened flowers.—clear all over pink.
   3 days open.—color similar to newly opened flower, maintains its color, does not fade. Flower (petal) color becomes progressively darker as the flower ages.
Reproductive organs: Stamens are moderately abundant, arranged regularly about the pistil. The filaments are fairly short and yellow in color. Anthers are well formed and yellow in color. Pollen is moderate. There is an average number of pistils. Styles are long and bunched and near-white in color. Ovaries are all enclosed in the calyx. Occasionally, there are hips and there are seed.
Foliage: Compound of seven leaflets; medium, leathery; semi-glossy. Leaflets are broad elliptical; apex acute; base - rounded. The color of the leaflets is medium green (can be as dark as Green 137A); underside of leaf is lighter green (138A).
   Rachis.—Medium strength; upper side smooth; grooved; underside may have several small thorns (or claws).
   Stipules.—Medium to long length; medium width; medium to short length narrow points which turn out usually at about 45 degrees or less. Color is similar to leaflets.
   Habit.—Bushy; upright; much branched. Plant height: 3 feet. Plant spread: 3 feet.
   Growth.—Vigorous; internodes medium.
   Canes.—Medium and sturdy.
   Main stems.—Color: Medium green, near Green Group 138A.
   Thorns.—Medium size and length, needle-shaped. Color: brown. Hairs — none.

The floribunda rose plant and its flowers heretofore described may vary in slight detail due to climate, soil and cultural conditions under which the cultivar may be grown. The present description is of the cultivar as grown at Visalia, Calif.

The following is claimed:

1. This invention relates to a new and distinct cultivar of floribunda rose, primarily identified by the pink color of its petals and double flowers.
